(19) United States
(12) Patent Application Publication
(10) Pub. No.: US 2018/0099000 A1
(43) Pub. Date: Apr. 12, 2018

(54) Method for the Prophylaxis or Treatment of Systemic Lupus Erythematosus and/or Lupus Nephritis

(71) Applicant: Millennium Pharmaceuticals, Inc., Cambridge, MA (US)

(72) Inventors: Toru Kawamura, Kanagawa (JP); Yasuahi Fujitani, San Diego, CA (US); Masayuki Takizawa, Kanagawa (JP)

(21) Appl. No.: 15/838,595

(22) Filed: Dec. 12, 2017

Related U.S. Application Data
(63) Continuation of application No. 15/026,417, filed on Mar. 31, 2016, now abandoned.
(60) Provisional application No. 61/886,403, filed on Oct. 3, 2013.

(51) Int. Cl. A61K 31/69; A61K 9/48; A61K 9/19; A61K 9/00

(52) U.S. Cl. CPC A61K 31/69; A61K 9/48; A61K 9/19; A61K 9/0053; A61K 9/0019

(57) ABSTRACT

The present invention provides a method for the prophylaxis or treatment of systemic lupus erythematosus and/or lupus nephritis in a patient in need thereof, which comprises administering an effective amount of [(1R)-1-({[2,5-dichlorobenzoyl)amino]acetyl}amino)-3-methylbutyl]boronic acid or a citric acid ester thereof, or a pharmaceutically acceptable salt thereof to the patient.

### TECHNICAL FIELD

The present invention relates to a method for the prophylaxis or treatment of systemic lupus erythematosus and/or lupus nephritis.

### BACKGROUND OF THE INVENTION

Systemic lupus erythematosus (SLE) is a chronic autoimmune disorder that has protean manifestations and follows a relapsing and remitting course. SLE can affect any organ system, but mainly involves the skin, joints, kidneys, and nervous system. Lupus nephritis (LN) is one of the most serious SLE complications since it is the major predictor of poor prognosis. While available therapies, such as steroid and immunosuppressants e.g. cyclophosphamide and mycophenolate, have improved the outcomes of patients with SLE and/or LN, there remains a significant unmet need for safe and more effective treatments (see Arthritis Care & Research, 63, 797-808 (2012)).

As compounds for the prophylaxis or treatment of autoimmune disease, the following compounds are disclosed.

Patent Documents 1-6 and non-patent Document 1 disclose a peptidic compound for the prophylaxis or treatment of autoimmune disease.

### DOCUMENT LIST

### Patent Document

Patent Document 1: WO 96/13266

Patent Document 2: WO 2009/020448

Patent Document 3: WO 2009/154737

Patent Document 4: WO 2010/036357

Patent Document 5: WO 2011/123502

Patent Document 6: WO 2012/1190567

### Non-Patent Document

Non-Patent Document 1: Nature Medicine, vol. 14, 748-755, 2008,

### SUMMARY OF THE INVENTION

### Problems to be Solved by the Invention

There is a demand for the development of a compound useful for the prophylaxis or treatment of systemic lupus erythematosus, lupus nephritis and the like, and has efficacy and low toxicity.

### Means of Solving the Problems

The present inventors have conducted intensive studies in an attempt to solve the above-mentioned problem and found that [(1R)-1-({[(2,5-dichlorobenzoyl)amino]acetyl}amino)-3-methylbutyl]boronic acid or a citric acid ester thereof, or a pharmaceutically acceptable salt thereof is useful for the prophylaxis or treatment of systemic lupus erythematosus, lupus nephritis and the like, and has efficacy and low toxicity. Based on this finding, the present inventors have conducted intensive studies and completed the present invention.

Accordingly, the present invention provides the following.

(1) A method for the prophylaxis or treatment of systemic lupus erythematosus and/or lupus nephritis in a patient in need thereof, which comprises administering an effective amount of a compound which is [(1R)-1-({[(2,5-dichlorobenzoyl)amino]acetyl}amino)-3-methylbutyl]boronic acid or a citric acid ester thereof, or a pharmaceutically acceptable salt thereof to the patient;
(2) the method of the above-mentioned (1), wherein the compound is 2,2′-{2-[(1R)-1-({[(2,5-dichlorobenzoyl)amino]acetyl}amino)-3-methylbutyl]-5-oxo-1,3,2-dioxaborolane-4,4-diyl}diacetic acid, 4-(R,S)-(carboxymethyl)-2-((R)-1-(2-(2,5-dichlorobenzamido)acetamido)-3-methylbutyl)-6-oxo-1,3,2-dioxaborinane-4-carboxylic acid, or
a mixture thereof;
(3) the method of the above-mentioned (1), wherein the compound is 2,2′-{2-[(1R)-1-({[(2,5-dichlorobenzoyl)amino]acetyl}amino)-3-methylbutyl]-5-oxo-1,3,2-dioxaborolane-4,4-diyl}diacetic acid;
(4) the method of the above-mentioned (1), wherein the compound is 4-(R,S)-(carboxymethyl)-2-((R)-1-(2-(2,5-dichlorobenzamido)acetamido)-3-methylbutyl)-6-oxo-1,3,2-dioxaborinane-4-carboxylic acid;
(5) A compound which is [(1R)-1-({[(2,5-dichlorobenzoyl)amino]acetyl}amino)-3-methylbutyl]boronic acid or a citric acid ester thereof, or a pharmaceutically acceptable salt thereof, for use in the prophylaxis or treatment of systemic lupus erythematosus and/or lupus nephritis;
(6) Use of a compound which is [(1R)-1-({[(2,5-dichlorobenzoyl)amino]acetyl}amino)-3-methylbutyl]boronic acid or a citric acid ester thereof, or a pharmaceutically acceptable salt thereof, for preparation of a medicament for the prophylaxis or treatment of systemic lupus erythematosus and/or lupus nephritis;
(7) A medicament for the prophylaxis or treatment of systemic lupus erythematosus and/or lupus nephritis, wherein the medicament comprises a compound which is [(1R)-1-({[(2,5-dichlorobenzoyl)amino]acetyl}amino)-3-methylbutyl]boronic acid or a citric acid ester thereof, or a pharmaceutically acceptable salt thereof;
and the like.

### Effect of the Invention

The present compound is useful for the prophylaxis or treatment of systemic lupus erythematosus, lupus nephritis and the like, and has efficacy and low toxicity.

### DETAILED DESCRIPTION OF THE INVENTION

Unless otherwise explicitly stated, the term “proteasome” used herein is intended to refer to constitutive proteasome, immunoproteasome, or both.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, the term “comprises” means “includes, but is not limited to”.

As used herein, the term “patient” means an animal, preferably a mammal, more preferably a human.

As used herein, the term “effective amount” means an amount that is sufficient upon appropriate administration to a patient (a) to cause a detectable decrease in the severity of the disorder or disease state being treated; (b) to ameliorate or alleviate the patient's symptoms of the disease or disorder; or (c) to slow or prevent advancement of, or otherwise stabilize or prolong stabilization of, the disorder or disease state being treated. It should also be understood that a specific dosage and treatment regimen for any particular patient will depend upon a variety of factors, including the activity of the specific compound employed, the age, body weight, general health, sex, and diet of the patient, time of administration, rate of excretion, drug combinations, the judgment of the treating physician, and the severity of the particular disease being treated.

As used herein, the term “treatment” means treating a patient having, or at risk of developing or experiencing a recurrence of the relevant disorder being treated, including suppression of progression of the relevant disorder being treated.

Unless otherwise stated, structures depicted herein are also meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

[(1R)-1-({[(2,5-dichlorobenzoyl)amino]acetyl}amino)-3-methylbutyl]boronic acid (alias; N²-(2,5-Dichlorobenzoyl)-N-[(1R)-1-(dihydroxyboranyl)-3-methylbutyl]glycinamide) [hereinafter to be referred to as compound (I)] is represented by the formula (I):

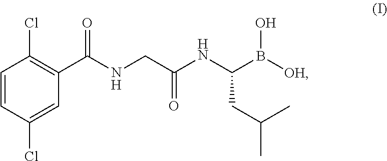 [SMILES: CC(C)C[C@H](NC(=O)CNC(=O)c1cc(Cl)ccc1Cl)B(O)O]

and disclosed in Olhava and Danca, U.S. Pat. No. 7,442,830 and WO 2009/020448, herein incorporated by reference in its entirety.

The dihydroxyboranyl group (—B(OH)₂) of compound (I) is optionally esterified with citric acid.

Prefereble examples of the citric acid ester of compound (I) include 2,2′-{2-[(1R)-1-({[(2,5-dichlorobenzoyl)amino]acetyl}amino)-3-methylbutyl]-5-oxo-1,3,2-dioxaborolane-4,4-diyl}diacetic acid (alias: 2,2′-{2-[(1R)-1-{[N-(2,5-dichlorobenzoyl)glycyl]amino}-3-methylbutyl]-5-oxo-1,3,2-dioxaborolane-4,4-diyl}diacetic acid) [hereinafter to be referred to as compound (Ia)], which is represented by the formula:

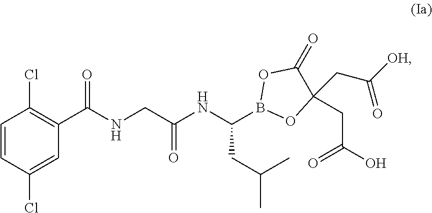 [SMILES: CC(C)C[C@H](NC(=O)CNC(=O)c1cc(Cl)ccc1Cl)B1OC(=O)C(CC(=O)O)(CC(=O)O)O1]

and 4-(R,S)-(carboxymethyl)-2-((R)-1-(2-(2,5-dichlorobenzamido)acetamido)-3-methylbutyl)-6-oxo-1,3,2-dioxaborinane-4-carboxylic acid (alias; 4-(carboxymethyl)-2-[(1R)-1-{[N-(2,5-dichlorobenzoyl)glycyl]amino}-3-methylbutyl]-6-oxo-1,3,2-dioxaborinane-4-carboxylic acid) [hereinafter to be referred to as compound (Ib)], which is represented by the formula:

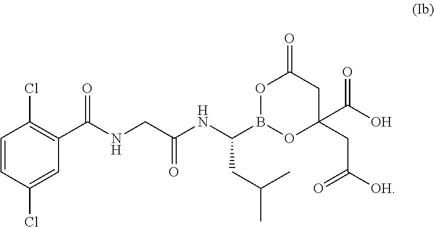 [SMILES: CC(C)C[C@H](NC(=O)CNC(=O)c1cc(Cl)ccc1Cl)B1OC(=O)CC(CC(=O)O)(C(=O)O)O1]

Compounds (Ia) and (Ib) are disclosed in WO 2009/154737, herein incorporated by reference in its entirety.

Unless otherwise stated, any crystalline form of compound (I) or a citric acid ester thereof is within the scope of the invention.

Compound (I) or a citric acid ester thereof (including compounds (Ia) and (Ib)) or a pharmaceutically acceptable salt thereof (hereinafter to be referred to as the compound of the present invention) is useful as a proteasome inhibitor for mammals (e.g., mouse, rat, hamster, rabbit, cat, dog, bovine, sheep, monkey, human, etc.). The compound of the present invention is used as a pharmaceutical agent such as an agent for the prophylaxis or treatment of diseases possibly influenced by proteasome (e.g., autoimmune disease and antibody-mediated disease).

Specifically, the compound of the present invention is useful for the prophylaxis or treatment of systemic lupus . erythematosus, lupus nephritis, rheumatoid arthritis, Sjogren's syndrome, psoriasis, ulcerative colitis, Crohn's disease, type 1 diabetes, myasthenia gravis, multiple sclerosis, idiopathic pulmonary fibrosis, cirrhosis, endomyocardial fibrosis, scleroderma/systemic sclerosis, antibody-mediated rejection, antibody-mediated rejection in organ transplantation, antibody-mediated rejection in kidney transplantation, antibody-mediated rejection in lung transplantation, antibody-mediated rejection in heart transplantation, antibody-mediated rejection in liver transplantation, antibody-mediated rejection in pancreas transplantation, graft versus host disease and the like.

The compound of the present invention is useful for desensitization therapy.

In preferable embodiments, the compound of the present invention (preferably the citric acid ester of compound (I), more preferably compound (Ia)) is used for the prophylaxis or treatment of systemic lupus erythematosus and/or lupus nephritis.

The present invention provides a method for the prophylaxis or treatment of systemic lupus erythematosus and/or lupus nephritis in a patient in need thereof, which comprises administering an effective amount of the compound of the present invention to the patient.

In some embodiments, the present invention provides a method for the prophylaxis or treatment of systemic lupus erythematosus and/or lupus nephritis in a patient in need thereof, which comprises administering an effective amount of compound (Ia) to the patient.

In some embodiments, the present invention provides a method for the prophylaxis or treatment of systemic lupus erythematosus and/or lupus nephritis in a patient in need thereof, which comprises administering an effective amount of compound (Ib) to the patient.

In some embodiments, the present invention provides a method for the prophylaxis or treatment of systemic lupus erythematosus and/or lupus nephritis in a patient in need thereof, which comprises administering an effective amount of a mixture of compound (Ia) and compound (Ib) to the patient.

The present invention provides the compound of the present invention for use in the prophylaxis or treatment of systemic lupus erythematosus and/or lupus nephritis.

In some embodiments, the present invention provides compound (Ia) for use in the prophylaxis or treatment of systemic lupus erythematosus and/or lupus nephritis.

In some embodiments, the present invention provides compound (Ib) for use in the prophylaxis or treatment of systemic lupus erythematosus and/or lupus nephritis.

In some embodiments, the present invention provides a mixture of compound (Ia) and compound (Ib) for use in the prophylaxis or treatment of systemic lupus erythematosus and/or lupus nephritis.

The present invention provides use of the compound of the present invention for preparation of a medicament for the prophylaxis or treatment of systemic lupus erythematosus and/or lupus nephritis.

In some embodiments, the present invention provides use of compound (Ia) for preparation of a medicament for the prophylaxis or treatment of systemic lupus erythematosus and/or lupus nephritis.

In some embodiments, the present invention provides use of compound (Ib) for preparation of a medicament for the prophylaxis or treatment of systemic lupus erythematosus and/or lupus nephritis.

In some embodiments, the present invention provides use of a mixture of compound (Ia) and compound (Ib) for preparation of a medicament for the prophylaxis or treatment of systemic lupus erythematosus and/or lupus nephritis.

The present invention provides a medicament for the prophylaxis or treatment of systemic lupus erythematosus and/or lupus nephritis, wherein the medicament comprises the compound of the present invention.

In some embodiments, the present invention provides a medicament for the prophylaxis or treatment of systemic lupus erythematosus and/or lupus nephritis, wherein the medicament comprises compound (Ia).

In some embodiments, the present invention provides a medicament for the prophylaxis or treatment of systemic lupus erythematosus and/or lupus nephritis, wherein the medicament comprises compound (Ib).

In some embodiments, the present invention provides a medicament for the prophylaxis or treatment of systemic lupus erythematosus and/or lupus nephritis, wherein the medicament comprises a mixture of compound (Ia) and compound (Ib).

The present invention provides the use of the compound of the present invention, for the preparation of a pharmaceutical composition (as described herein) for the treatment of systemic lupus erythematosus and/or lupus nephritis.

In some embodiments, the present invention provides the use of compound (Ia), for the preparation of a pharmaceutical composition for the treatment of systemic lupus erythematosus and/or lupus nephritis.

In some embodiments, the present invention provides the use of compound (Ib), for the preparation of a pharmaceutical composition for the treatment of systemic lupus erythematosus and/or lupus nephritis.

In some embodiments, the present invention provides the use of a mixture of compound (Ia) and compound (Ib), for the preparation of a pharmaceutical composition for the treatment of systemic lupus erythematosus and/or lupus nephritis.

The present invention provides the use of an effective amount of the compound of the present invention for the treatment of systemic lupus erythematosus and/or lupus nephritis.

In some embodiments, the present invention provides the use of an effective amount of compound (Ia) for the treatment of systemic lupus erythematosus and/or lupus nephritis.

In some embodiments, the present invention provides the use of an effective amount of compound (Ib) for the treatment of systemic lupus erythematosus and/or lupus nephritis.

In some embodiments, the present invention provides the use of an effective amount of a mixture of compound (Ia) and compound (Ib) for the treatment of systemic lupus erythematosus and/or lupus nephritis.

In some embodiments, the compound of the present invention or a pharmaceutical composition thereof is administered orally.

In certain embodiments, compound (Ia) or a pharmaceutical composition thereof is administered orally. In certain such embodiments, compound (Ia) or a pharmaceutical composition thereof is administered in one or more capsules.

In some embodiments, the compound of the present invention or a pharmaceutical composition thereof is administered intravenously.

In certain embodiments, compound (Ia) or a pharmaceutical composition thereof is administered intravenously.

While the dose varies depending on the target disease, symptom, subject of administration, administration method and the like, for oral administration as a therapeutic agent for systemic lupus erythematosus and/or lupus nephritis, for example, it is generally about 0.01-100 mg/kg body weight, preferably 0.05-30 mg/kg body weight, more preferably 0.5-10 mg/kg body weight, as one dose of the compound of the present invention, which is, for example, administered once to 3 times a day, on a weekly schedule, on a twice-weekly schedule and the like.

In some embodiments, the compound of the present invention or a pharmaceutical composition thereof is administered on a weekly schedule.

In some embodiments, compound (Ia) or a pharmaceutical composition thereof is administered on a weekly schedule.

In some embodiments, the compound of the present invention or a pharmaceutical composition thereof is administered on days 1, 8, and 15 of a 28-day cycle.

In some embodiments, compound (Ia) or a pharmaceutical composition thereof is administered on days 1, 8, and 15 of a 28-day cycle.

In some embodiments, the compound of the present invention or a pharmaceutical composition thereof is administered on a twice-weekly schedule.

In some embodiments, compound (Ia) or a pharmaceutical composition thereof is administered on a twice-weekly schedule.

In some embodiments, the compound of the present invention or a pharmaceutical composition thereof is administered on days 1, 4, 8, and 11 of a 21-day cycle.

In some embodiments, compound (Ia) or a pharmaceutical composition thereof is administered on days 1, 4, 8, and 11 of a 21-day cycle.

In some embodiments, the compound of the present invention or a pharmaceutical composition thereof is administered in conjunction with the other therapeutic modality.

In certain embodiments, compound (Ia) or a pharmaceutical composition thereof is administered in conjunction with the other therapeutic modality.

In certain such embodiments, the other therapeutic modality is one that is normally administered to patients with systemic lupus erythematosus and/or lupus nephritis.

In some such embodiments, the other therapeutic modality is radiotherapy or plasmapheresis.

In some such embodiments, the other therapeutic modality is the other therapeutic agent.

In some such embodiments, the other therapeutic modality is radiotherapy and one or more therapeutic agents.

In the above embodiments, the other therapeutic agent may be administered in the same dosage form or as a separate dosage form. When administered as a separate dosage form, the other therapeutic agent may be administered prior to, at the same time as, or following administration of the compound of the present invention or a pharmaceutical composition thereof.

The compound of the present invention can be used together with other drugs for the prophylaxis or treatment of various diseases.

For example, when the compound of the present invention is used as a proteasome inhibitor, it can be used together with the following drugs.

(1) non-steroidal antiinflammatory drugs (NSAIDs)

### (i) Classical NSAIDs

alcofenac, aceclofenac, sulindac, tolmetin, etodolac, fenoprofen, thiaprofenic acid, meclofenamic acid, meloxicam, tenoxicam, lornoxicam, nabumeton, acetaminophen, phenacetin, ethenzamide, sulpyrine, antipyrine, migrenin, aspirin, mefenamic acid, flufenamic acid, diclofenac sodium, loxoprofen sodium, phenylbutazone, indomethacin, ibuprofen, ketoprofen, naproxen, oxaprozin, flurbiprofen, fenbufen, pranoprofen, floctafenine, piroxicam, epirizole, tiaramide hydrochloride, zaltoprofen, gabexate mesylate, camostat mesylate, ulinastatin, colchicine, probenecid, sulfinpyrazone, benzbromarone, allopurinol, sodium aurothiomalate, hyaluronate sodium, sodium salicylate, morphine hydrochloride, salicylic acid, atropine, scopolamine, morphine, pethidine, levorphanol, oxymorphone or a salt thereof and the like.

(ii) cyclooxygenase inhibitor (COX-1 selective inhibitor, COX-2 selective inhibitor and the like)

salicylic acid derivatives (e.g., celecoxib, aspirin), etoricoxib, valdecoxib, diclofenac, indomethacin, loxoprofen and the like.

(iii) nitric oxide-releasing NSAIDs
(2) disease-modifying anti-rheumatic drugs (DMARDs)
(i) Gold preparation

auranofin and the like.

(ii) penicillamine

D-penicillamine

(iii) aminosalicylic acid preparation

sulfasalazine, mesalazine, olsalazine, balsalazide and the like.

(iv) antimalarial drug

chloroquine and the like.

(v) pyrimidine synthesis inhibitor

leflunomide and the like.

(vi) prograf
(3) anti-cytokine drug
(I) protein drug
(i) TNF inhibitor

etanercept, infliximab, adalimumab, certolizumab pegol, golimumab, PASSTNF-α, soluble TNF-α receptor, TNF-α binding protein, anti-TNF-α antibody and the like.

(ii) interleukin-1 inhibitor

anakinra (interleukin-1 receptor antagonist), soluble interleukin-1 receptor and the like.

(iii) interleukin-6 inhibitor

tocilizumab (anti-interleukin-6 receptor antibody), anti-interleukin-6 antibody and the like.

(iv) interleukin-10 drug

interleukin-10 and the like.

(v) interleukin-12/23 inhibitor

ustekinumab, briakinumab (anti-interleukin-12/23 antibody) and the like.

(vi) B cell activation inhibitor

rituximab, belimumab and the like.

(vii) co-stimulatory molecules-related protein preparation

abatacept and the like.

(II) non-protein drug
(i) MAPK inhibitor

BMS-582949 and the like.

(ii) gene modulator

inhibitor of molecule involved in signal transduction, such as NF-κ, NF-κB, IKK-1, IKK-2, AP-1 and the like, and the like.

(iii) cytokine production inhibitor

iguratimod, tetomilast and the like.

(iv) TNF-α converting enzyme inhibitor
(v) interleukin-1β converting enzyme inhibitor

VX-765 and the like.

(vi) interleukin-6 antagonist

HMPL-004 and the like.

(vii) interleukin-8 inhibitor

IL-8 antagonist, CXCR1 & CXCR2 antagonist, reparixin and the like.

(viii) chemokine antagonist

CCR9 antagonist (CCX-282, CCX-025), MCP-1 antagonist and the like.

(ix) interleukin-2 receptor antagonist

denileukin, diftitox and the like.

(x) therapeutic vaccines

TNF-α vaccine and the like.

(xi) gene therapy drug

gene therapy drugs aiming at promoting the expression of gene having an anti-inflammatory action such as interleukin-4, interleukin-10, soluble interleukin-1 receptor, soluble TNF-α receptor and the like.

(xii) antisense compound

ISIS-104838 and the like.

(4) integrin inhibitor

natalizumab, vedolizumab, AJM300, TRK-170, E-6007 and the like.

(5) immunomodulator (immunosuppressant)

methotrexate, cyclophosphamide, MX-68, atiprimod dihydrochloride, BMS-188667, CKD-461, rimexolone, cyclosporine, tacrolimus, gusperimus, azathiopurine, antilymphocyte serum, freeze-dried sulfonated normal immunoglobulin, erythropoietin, colony stimulating factor, interleukin, interferon, intravenous immunoglobulin, anti-thymocyte globulin, RSLV-132 and the like.

(6) proteasome inhibitor

bortezomib and the like.

(7) JAK inhibitor

tofacitinib and the like.

(8) steroid

dexamethasone, hexestrol, methimazole, betamethasone, triamcinolone, triamcinolone acetonide, fluocinonide, fluocinolone acetonide, predonisolone, methylpredonisolone, cortisone acetate, hydrocortisone, fluorometholone, beclomethasone dipropionate, estriol and the like.

(9) angiotensin converting enzyme inhibitor

enalapril, captopril, ramipril, lisinopril, cilazapril, perindopril and the like.

(10) angiotensin II receptor antagonist

candesartan, candesartan cilexetil (TCV-116), valsartan, irbesartan, olmesartan, eprosartan and the like.

(11) diuretic drug

hydrochlorothiazide, spironolactone, furosemide, indapamide, bendrofluazide, cyclopenthiazide and the like.

(12) cardiotonic drug

digoxin, dobutamine and the like.

(13) β receptor antagonist

carvedilol, metoprolol, atenolol and the like.

(14) Ca sensitizer

MCC-135 and the like.

(15) Ca channel antagonist

nifedipine, diltiazem, verapamil and the like.

(16) anti-platelet drug, anticoagulator

heparin, aspirin, warfarin and the like.

(17) HMG-CoA reductase inhibitor

atorvastatin, simvastatin and the like.

(18) contraceptive
(i) sex hormone or derivatives thereof

gestagen or a derivative thereof (e.g., progesterone, 17α-hydroxy progesterone, medroxyprogesterone, medroxyprogesterone acetate, norethisterone, norethisterone enanthate, norethindrone, norethindrone acetate, norethynodrel, levonorgestrel, norgestrel, ethynodiol diacetate, desogestrel, norgestimate, gestodene, progestin, etonogestrel, drospirenone, dienogest, trimegestone, nestorone, chlormadinone acetate, mifepristone, nomegestrol acetate, Org-30659, TX-525, EMM-310525) or a combination agent of a gestagen or a derivative thereof and an estrogen or a derivative thereof (estradiol, estradiol benzoate, estradiol cypionate, estradiol dipropionate, estradiol enanthate, estradiol hexahydrobenzoate, estradiol phenylpropionate, estradiol undecanoate, estradiol valerate, estrone, ethinylestradiol, mestranol) and the like.

(ii) antiestrogen

ormeloxifene, mifepristone, Org-33628 and the like.

(iii) spermatocide

ucarcide and the like.

(19) others
(i) T cell inhibitors
(ii) inosine monophosphate dehydrogenase (IMPDH) inhibitor mycophenolate mofetil and the like.
(iii) adhesion molecule inhibitor

ISIS-2302, selectin inhibitor, ELAM-1, VCAM-1, ICAM-1 and the like.

(iv) thalidomide
(v) cathepsin inhibitor
(vi) matrix metalloprotease (MMPs) inhibitor

V-85546 and the like.

(vii) glucose-6-phosphate dehydrogenase inhibitor
(viii) Dihydroorotate dehydrogenase (DHODH) inhibitor
(ix) phosphodiesterase IV (PDE IV) inhibitor

roflumilast, CG-1088 and the like.

(x) phospholipase A₂ inhibitor
(xi) iNOS inhibitor

VAS-203 and the like.

(xii) microtubule stimulating drug

paclitaxel and the like.

(xiii) microtubule inhibitor

reumacon and the like.

(xiv) MHC class II antagonist
(xv) prostacyclin agonist iloprost and the like.
(xvi) CD4 antagonist

zanolimumab and the like.

(xvii) CD23 antagonist
(xviii) LTB4 receptor antagonist

DW-1305 and the like.

(xix) 5-lipoxygenase inhibitor

zileuton and the like.

(xx) cholinesterase inhibitor galanthamine and the like.
(xxi) tyrosine kinase inhibitor

Tyk2 inhibitor (WO 2010/142752) and the like.

(xxii) cathepsin B inhibitor
(xxiii) adenosine deaminase inhibitor pentostatin and the like.
(xxiv) osteogenesis stimulator
(xxv) dipeptidylpeptidase inhibitor
(xxvi) collagen agonist
(xxvii) capsaicin cream
(xxviii) hyaluronic acid derivative synvisc (hylan G-F 20), orthovisc and the like.
(xxix) glucosamine sulfate
(xxx) amiprilose
(xxxi) CD-20 inhibitor rituximab, ibritumomab, tositumomab, ofatumumab and the like.
(xxxii) BAFF inhibitor belimumab, tabalumab, atacicept, A-623 and the like.
(xxxiii) CD52 inhibitor

alemtuzumab and the like.

Other concomitant drugs besides the above-mentioned include, for example, antibacterial agent, antifungal agent, antiprotozoal agent, antibiotic, antitussive and expectorant drug, sedative, anesthetic, antiulcer drug, antiarrhythmic agent, hypotensive diuretic drug, anticoagulant, tranquilizer, antipsychotic, antitumor drug, hypolipidemic drug, muscle relaxant, anticonvulsant, antidepressant, antiallergic drug, cardiac stimulants, therapeutic drug for arrhythmia, vasodilator, vasoconstrictor, hypotensive diuretic, therapeutic drug for diabetes, antinarcotic, vitamin, vitamin derivative, antiasthmatic, therapeutic agent for pollakisuria/anischuria, therapeutic agent for atopic dermatitis, therapeutic agent for allergic rhinitis, hypertensor, endotoxin-antagonist or -antibody, signal transduction inhibitor, inhibitor of inflammatory mediator activity, antibody to inhibit inflammatory mediator activity, inhibitor of anti-inflammatory mediator activity, antibody to inhibit anti-inflammatory mediator activity and the like. Specific examples thereof include the following.

(1) Antibacterial agent
(i) sulfa drug

sulfamethizole, sulfisoxazole, sulfamonomethoxine, sulfamethizole, salazosulfapyridine, silver sulfadiazine and the like.

(ii) quinolone antibacterial agent

nalidixic acid, pipemidic acid trihydrate, enoxacin, norfloxacin, ofloxacin, tosufloxacin tosilate, ciprofloxacin hydrochloride, lomefloxacin hydrochloride, sparfloxacin, fleroxacin and the like.

(iii) antiphthisic

isoniazid, ethambutol (ethambutol hydrochloride), p-aminosalicylic acid (calcium p-aminosalicylate), pyrazinamide, ethionamide, protionamide, rifampicin, streptomycin sulfate, kanamycin sulfate, cycloserine and the like.

(iv) antiacidfast bacterium drug

diaphenylsulfone, rifampicin and the like.

(v) antiviral drug

idoxuridine, acyclovir, vidarabine, gancyclovir and the like.

(vi) anti-HIV agent

zidovudine, didanosine, zalcitabine, indinavir sulfate ethanolate, ritonavir and the like.

(vii) antispirochetele
(viii) antibiotic

tetracycline hydrochloride, ampicillin, piperacillin, gentamicin, dibekacin, kanendomycin, lividomycin, tobramycin, amikacin, fradiomycin, sisomicin, tetracycline, oxytetracycline, rolitetracycline, doxycycline, ampicillin, piperacillin, ticarcillin, cephalothin, cephapirin, cephaloridine, cefaclor, cephalexin, cefroxadine, cefadroxil, cefamandole, cefotoam, cefuroxime, cefotiam, cefotiam hexetil, cefuroxime axetil, cefdinir, cefditoren pivoxil, ceftazidime, cefpiramide, cefsulodin, cefmenoxime, cefpodoxime proxetil, cefpirome, cefozopran, cefepime, cefsulodin, cefmenoxime, cefmetazole, cefminox, cefoxitin, cefbuperazone, latamoxef, flomoxef, cefazolin, cefotaxime, cefoperazone, ceftizoxime, moxalactam, thienamycin, sulfazecin, aztreonam or a salt thereof, griseofulvin, lankacidin-group [Journal of Antibiotics (J. Antibiotics), 38, 877-885(1985)], azole compound [2-[(1R,2R)-2-(2,4-difluorophenyl)-2-hydroxy-1-methyl-3-(1H-1,2,4-triazol-1-yl)propyl]-4-[4-(2,2,3,3-tetrafluoropropoxy)phenyl]-3(2H,4H)-1,2,4-triazolone, fluconazole, itraconazole and the like] and the like.

(2) antifungal agent
(i) polyethylene antibiotic (e.g., amphotericin B, nystatin, trichomycin, etc.)
(ii) griseofulvin, pyrrolnitrin and the like.
(iii) cytosine metabolism antagonist (e.g., flucytosine)
(iv) imidazole derivative (e.g., econazole, clotrimazole, miconazole nitrate, bifonazole and croconazole)
(v) triazole derivative (e.g. fluconazole and itraconazole)
(vi) thiocarbamic acid derivative (e.g. trinaphthol), and the like.
(3) antiprotozoal agent

metronidazole, tinidazole, diethylcarbamazine citrate, quinine hydrochloride, quinine sulfate and the like.

(4) antitussive and expectorant drug

ephedrine hydrochloride, noscapine hydrochloride, codeine phosphate, dihydrocodeine phosphate, isoproterenol hydrochloride, ephedrine hydrochloride, methylephedrine hydrochloride, noscapine hydrochloride, alloclamide, chlophedianol, picoperidamine, cloperastine, protokylol, isoproterenol, salbutamol, terbutaline, oxymetebanol, morphine hydrochloride, dextromethorfan hydrobromide, oxycodone hydrochloride, dimemorphan phosphate, tipepidine hibenzate, pentoxyverine citrate, clofedanol hydrochloride, benzonatate, guaifenesin, bromhexine hydrochloride, ambroxol hydrochloride, acetylcysteine, ethyl cysteine hydrochloride, carbocysteine and the like.

(5) sedative

chlorpromazine hydrochloride, atropine sulfate, phenobarbital, barbital, amobarbital, pentobarbital, thiopental sodium, thiamylal sodium, nitrazepam, estazolam, flurazepam, haloxazolam, triazolam, flunitrazepam, bromovalerylurea, chloral hydrate, triclofos sodium and the like.

(6) anesthetic
(6-1) local anesthetic

cocaine hydrochloride, procaine hydrochloride, lidocaine, dibucaine hydrochloride, tetracaine hydrochloride, mepivacaine hydrochloride, bupivacaine hydrochloride, oxybuprocaine hydrochloride, ethyl aminobenzoate, oxethazaine and the like.

(6-2) general anesthetic
(i) inhalation anesthetic (e.g., ether, halothane, nitrous oxide, isoflurane, enflurane, etc.)
(ii) intravenous anesthetic (e.g., ketamine hydrochloride, droperidol, thiopental sodium, thiamylal sodium, pentobarbital, etc.) and the like.
(7) antiulcer drug

histidine hydrochloride, lansoprazole, metoclopramide, pirenzepine, cimetidine, ranitidine, famotidine, urogastrone, oxethazaine, proglumide, omeprazole, sucralfate, sulpiride, cetraxate, gefarnate, aldioxa, teprenone, prostaglandin and the like.

(8) antiarrhythmic agent
(i) Na channel blocker (e.g., quinidine, procainamide, disopyramide, ajmaline, lidocaine, mexiletine, phenytoin)
(ii) β-blocker (e.g., propranolol, alprenolol, bufetolol hydrochloride, oxprenolol, atenolol, acebutolol, metoprolol, bisoprolol, pindolol, carteolol, arotinolol hydrochloride)
(iii) K channel blocker (e.g., amiodarone)
(iv) Ca channel blocker (e.g., verapamil, diltiazem) and the like.
(9) hypotensive diuretic drug

hexamethonium bromide, clonidine hydrochloride, hydrochlorothiazide, trichlormethiazide, furosemide, ethacrynic acid, bumetanide, mefruside, azosemide, spironolactone, potassium canrenoate, triamterene, amiloride, acetazolamide, D-mannitol, isosorbide, aminophylline, and the like.

(10) anticoagulant

heparin sodium, sodium citrate, activated protein C, tissue factor pathway inhibitor, antithrombin III, dalteparin sodium, warfarin potassium, argatroban, gabexate, sodium citrate, ozagrel sodium, ethyl icosapentate, beraprost sodium, alprostadil, ticlopidine hydrochloride, pentoxifylline, dipyridamole, tisokinase, urokinase, streptokinase and the like.

(11) tranquilizer

diazepam, lorazepam, oxazepam, chlordiazepoxide, medazepam, oxazolam, cloxazolam, clotiazepam, bromazepam, etizolam, fludiazepam, hydroxyzine and the like.

(12) antipsychotic

chlorpromazine hydrochloride, prochlorperazine, trifluoperazine, thioridazine hydrochloride, perphenazine maleate, fluphenazine enanthate, prochlorperazine maleate, levomepromazine maleate, promethazine hydrochloride, haloperidol, bromperidol, spiperone, reserpine, clocapramine hydrochloride, sulpiride, zotepine and the like.

(13) antitumor drug

6-O-(N-chloroacetylcarbamoyl)fumagillol, bleomycin, methotrexate, actinomycin D, mitomycin C, daunorubicin, adriamycin, neocarzinostatin, cytosine arabinoside, fluorouracil, tetrahydrofuryl-5-fluorouracil, picibanil, lentinan, levamisole, bestatin, azimexon, glycyrrhizin, doxorubicin hydrochloride, aclarubicin hydrochloride, bleomycin hydrochloride, peplomycin sulfate, vincristine sulfate, vinblastine sulfate, irinotecan hydrochloride, cyclophosphamide, melphalan, busulfan, thiotepa, procarbazine hydrochloride, cisplatin, azathioprine, mercaptopurine, tegafur, carmofur, cytarabine, methyltestosterone, testosterone propionate, testosterone enanthate, mepitiostane, fosfestrol, chlormadinone acetate, leuprorelin acetate, buserelin acetate and the like.

(14) antihypolipidemic drug

clofibrate, ethyl 2-chloro-3-[4-(2-methyl-2-phenylpropoxy)phenyl]propionate [Chemical and Pharmaceutical Bulletin (Chem. Pharm. Bull), 38, 2792-2796 (1990)], pravastatin, simvastatin, probucol, bezafibrate, clinofibrate, nicomol, cholestyramine, dextran sulfate sodium and the like.

(15) muscle relaxant

pridinol, tubocurarine, pancuronium, tolperisone hydrochloride, chlorphenesin carbamate, baclofen, chlormezanone, mephenesin, chlorzoxazone, eperisone, tizanidine and the like.

(16) anticonvulsant

phenytoin, ethosuximide, acetazolamide, chlordiazepoxide, trimethadione, carbamazepine, phenobarbital, primidone, sulthiame, sodium valproate, clonazepam, diazepam, nitrazepam and the like.

(17) antidepressant

imipramine, clomipramine, noxiptiline, phenelzine, amitriptyline hydrochloride, nortriptyline hydrochloride, amoxapine, mianserin hydrochloride, maprotiline hydrochloride, sulpiride, fluvoxamine maleate, trazodone hydrochloride and the like.

(18) antiallergic drug

diphenhydramine, chlorpheniramine, tripelennamine, metodilamine, clemizole, diphenylpyraline, methoxyphenamine, sodium cromoglicate, tranilast, repirinast, amlexanox, ibudilast, ketotifen, terfenadine, mequitazine, azelastine hydrochloride, epinastine, ozagrel hydrochloride, pranlukast hydrate, seratrodast and the like.

(19) cardiac

trans-π-oxocamphor, terephyllol, aminophylline, etilefrine, dopamine, dobutamine, denopamine, aminophylline, bencirin, amrinone, pimobendan, ubidecarenone, digitoxin, digoxin, methyldigoxin, lanatoside C, G-strophanthin and the like.

(20) vasodilator

oxyfedrine, diltiazem, tolazoline, hexobendine, bamethan, clonidine, methyldopa, guanabenz and the like.

(21) vasoconstrictor

dopamine, dobutamine denopamine and the like.

(22) hypotensive diuretic

Hexamethonium bromide, pentolinium, mecamylamine, ecarazine, clonidine, diltiazem, nifedipine and the like.

(23) antidiabetic drug

tolbutamide, chlorpropamide, acetohexamide, glibenclamide, tolazamide, acarbose, epalrestat, troglitazone, glucagon, glymidine, glipizide, phenformin, buformin, metformin and the like.

(24) antinarcotic

levallorphan, nalorphine, naloxone or a salt thereof and the like.

(25) liposoluble vitamins
(i) vitamin A: vitamin A₁, vitamin A₂ and retinol palmitate
(ii) vitamin D: vitamin D₁, D₂, D₃, D₄ and D₅
(iii) vitamin E: α-tocopherol, β-tocopherol, γ-tocopherol, δ-tocopherol, dl-α-tocopherol nicotinate
(iv) vitamin K: vitamin K₁, K₂, K₃ and K₄
(v) folic acid (vitamin M) and the like.
(26) vitamin derivative

various derivatives of vitamins, for example, vitamin D₃ derivatives such as 5,6-trans-cholecalciferol, 2,5-hydroxycholecalciferol, 1-α-hydroxycholecalciferol and the like, vitamin D₂ derivatives such as 5,6-trans-ergocalciferol and the like, and the like.

(27) antiasthmatic

isoprenaline hydrochloride, salbutamol sulfate, procaterol hydrochloride, terbutaline sulfate, trimetoquinol hydrochloride, tulobuterol hydrochloride, orciprenaline sulfate, fenoterol hydrobromide, ephedrine hydrochloride, ipratropium bromide, oxitropium bromide, flutropium bromide, theophylline, aminophylline, sodium cromoglicate, tranilast, repirinast, amlexanox, ibudilast, ketotifen, terfenadine, mequitazine, azelastine, epinastine, ozagrel hydrochloride, pranlkast hydrate, seratrodast, dexamethasone, prednisolone, hydrocortisone, hydrocortisone sodium succinate, beclometasone dipropionate and the like.

(28) therapeutic agent for pollakisuria/anischuria

flavoxate hydrochloride and the like.

(29) therapeutic agent for atopic dermatitis

sodium cromoglicate and the like.

(30) therapeutic agent for allergic rhinitis

sodium cromoglicate, chlorpheniramine maleate, alimemazine tartrate, clemastine fumarate, homochlorcyclizine hydrochloride, fexofenadine, mequitazine and the like.

(31) hypertensive drug

dopamine, dobutamine, denopamine, digitoxin, digoxin, methyldigoxin, lanatoside C, G-strophanthin and the like.

### (32) Others

hydroxycam, diacerein, megestrol acetate, nicergoline, prostaglandins and the like.

### Formulation and Administration

If a pharmaceutically acceptable salt of compound (I) or a citric acid ester thereof is utilized in these compositions, the salt preferably is derived from an inorganic or organic acid or base. For reviews of suitable salts, see, e.g., Berge et al, J. Pharm. Sci. 66:1-19 (1977) and Remington: The Science and Practice of Pharmacy, 20th Ed., ed. A. Gennaro, Lippincott Williams & Wilkins, 2000.

Non-limiting examples of suitable acid addition salts include the following: acetate, adipate, alginate, aspartate, benzoate, benzene sulfonate, bisulfate, butyrate, citrate, camphorate, camphor sulfonate, cyclopentanepropionate, digluconate, dodecylsulfate, ethanesulfonate, fumarate, lucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate.

Suitable base addition salts include, without limitation, ammonium salts, alkali metal salts, such as lithium, sodium and potassium salts; alkaline earth metal salts, such as calcium and magnesium salts; other multivalent metal salts, such as zinc salts; salts with organic bases, such as dicyclohexylamine, N-methyl-D-glucamine, t-butylamine, ethylene diamine, ethanolamine, and choline; and salts with amino acids such as arginine, lysine, and so forth.

The pharmaceutical composition comprises the compound of the present invention and a pharmaceutically acceptable carrier.

The term “pharmaceutically acceptable carrier” is used herein to refer to a material that is compatible with a recipient subject, preferably a mammal, more preferably a human, and is suitable for delivering an active agent to the target site without terminating the activity of the agent. The toxicity or adverse effects, if any, associated with the carrier preferably are commensurate with a reasonable risk/benefit ratio for the intended use of the active agent.

The terms “carrier”, “adjuvant”, or “vehicle” are used interchangeably herein, and include any and all solvents, diluents, and other liquid vehicles, dispersion or suspension aids, surface active agents, pH modifiers, isotonic agents, thickening or emulsifying agents, preservatives, solid binders, lubricants and the like, as suited to the particular dosage form desired. Remington: The Science and Practice of Pharmacy, 20th Ed., ed. A. Gennaro, Lippincott Williams & Wilkins, 2000 discloses various carriers used in formulating pharmaceutically acceptable compositions and known techniques for the preparation thereof. Except insofar as any conventional carrier medium is incompatible with the compound of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, carbonates, magnesium hydroxide and aluminum hydroxide, glycine, sorbic acid, or potassium sorbate, partial glyceride mixtures of saturated vegetable fatty acids, water, pyrogen-free water, salts or electrolytes such as protamine sulfate, disodium hydrogen phosphate, potassium hydrogen phosphate, sodium chloride, and zinc salts, colloidal silica, magnesium trisilicate, polyvinyl pyrrolidone, polyacrylates, waxes, polyethylene-polyoxypropylene-block polymers, wool fat, sugars such as lactose, glucose, sucrose, and mannitol, starches such as corn starch and potato starch, cellulose and its derivatives such as sodium carboxymethyl cellulose, ethyl cellulose and cellulose acetate, powdered tragacanth; malt, gelatin, talc, excipients such as cocoa butter and suppository waxes, oils such as peanut oil, cottonseed oil, safflower oil, sesame oil, olive oil, corn oil and soybean oil, glycols such as propylene glycol and polyethylene glycol, esters such as ethyl oleate and ethyl laurate, agar, alginic acid, isotonic saline, Ringer's solution, alcohols such as ethanol, isopropyl alcohol, hexadecyl alcohol, and glycerol, cyclodextrins such as hydroxypropyl beta-cyclodextrin and sulfobutylether beta-cyclodextrin, lubricants such as sodium lauryl sulfate and magnesium stearate, petroleum hydrocarbons such as mineral oil and petrolatum. Coloring agents, releasing agents, coating agents, sweetening, flavoring and perfuming agents, preservatives and antioxidants can also be present in the composition, according to the judgment of the formulator.

The pharmaceutical composition of the invention can be manufactured by methods well known in the art such as conventional granulating, mixing, dissolving, encapsulating, lyophilizing, or emulsifying processes, among others. The pharmaceutical composition may be produced in various forms, including granules, precipitates, or particulates, powders, including freeze dried, rotary dried or spray dried powders, amorphous powders, tablets, capsules, syrup, suppositories, injections, emulsions, elixirs, suspensions or solutions.

The pharmaceutical composition of the invention is formulated for pharmaceutical administration to a mammal, preferably a human being. Such pharmaceutical composition of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the pharmaceutical composition is administered orally, intravenously, or subcutaneously. The pharmaceutical composition may be designed to be short-acting, fast-releasing, or long-acting. Still further, the pharmaceutical composition can be administered in a local rather than systemic means.

Liquid dosage forms for oral administration include, but are not limited to, pharmaceutically acceptable emulsions, microemulsions, solutions, suspensions, syrups and elixirs. In addition to the compound of the present invention, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, cyclodextrins, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. Compositions formulated for parenteral administration may be injected by bolus injection or by timed push, or may be administered by continuous infusion.

Solid dosage forms for oral administration include capsules, tablets, pills, powders, and granules. In such solid dosage forms, the compound of the present invention is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents such as phosphates or carbonates.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

In some embodiments, the compound of the present invention or a pharmaceutical composition thereof is administered orally.

In certain embodiments, compound (Ia) or a pharmaceutical composition thereof is administered orally.

In some such embodiments, a pharmaceutical composition comprising compound (Ia) is prepared in gelatin capsules as described in WO 2009/154737, herein incorporated by reference in its entirety.

In some such embodiments, the pharmaceutical composition comprises compound (Ia), a filler, optionally a lubricant, optionally a flow-aid and optionally a buffer.

In some such embodiments, the pharmaceutical composition comprises compound (Ia), a filler, a lubricant, and a flow-aid.

In the above embodiments, the pharmaceutical composition comprises about 0.2% to about 12% of compound (Ia), or a crystalline form thereof, about 76.5% to about 99.8% of a filler, optionally up to about 1.5% of a lubricant, and optionally up to about 5% of a flow-aid. The oral pharmaceutical compositions can be prepared by methods described in Elliott et al., WO 2009/154737, herein incorporated by reference in its entirety.

The compound of the present invention can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the compound of the present invention may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of the compound of the present invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of the compound of the present invention to the body. Such dosage forms can be made by dissolving or dispensing the compound of the present invention in the proper medium. Absorption enhancers can also be used to increase the flux of the compound of the present invention across the skin. The rate can be controlled by either providing a rate controlling membrane or by dispersing the compound of the present invention in a polymer matrix or gel.

In some embodiments, the compound of the present invention or a pharmaceutical composition thereof is administered intravenously. In some such embodiments, the compound of the present invention or a pharmaceutical composition thereof can be prepared in the form of a lyophilized powder, as described in WO 02/059131, herein incorporated by reference in its entirety or WO 2009/154737, herein incorporated by reference in its entirety. In some embodiments, the lyophilized powder also comprises free boronic acid-complexing agent. Preferably, the free boronic acid-complexing agent and compound (I) are present in the mixture in a molar ratio ranging from about 0.5:1 to about 100:1, more preferably from about 5:1 to about 100:1. In various embodiments, the lyophilized powder comprises free boronic acid-complexing agent and the corresponding boronate ester in a molar ratio ranging from about 10:1 to about 100:1, from about 20:1 to about 100:1, or from about 40:1 to about 100:1.

In some embodiments, the lyophilized powder comprises boronic acid-complexing agent and compound (I), substantially free of other components. However, the pharmaceutical composition can further comprise one or more other pharmaceutically acceptable excipients, carriers, diluents, fillers, salts, buffers, bulking agents, stabilizers, solubilizers, and other materials well known in the art. The preparation of pharmaceutically acceptable formulations containing these materials is described in, e.g., Remington: The Science and Practice of Pharmacy, 20th Ed., ed. A. Gennaro, Lippincott Williams & Wilkins, 2000, or latest edition. In some embodiments, the pharmaceutical composition comprises the compound of the present invention, a bulking agent, and a buffer. In some embodiments, the pharmaceutical composition comprises compound (Ia) or a pharmaceutically acceptable salt thereof, a bulking agent, and a buffer.

The lyophilized powder comprising the compound of the present invention can be prepared according to the methods described in WO 02/059131, herein incorporated by reference in its entirety or WO 2009/154737, herein incorporated by reference in its entirety. In some embodiments, the method for preparing the lyophilized powder comprises: (a) preparing an aqueous mixture comprising compound (I) and a boronic acid-complexing agent; and (b) lyophilizing the mixture. In some embodiments, the method for preparing the lyophilized powder comprises: (a) preparing an aqueous mixture comprising compounds (Ia), a bulking agent, and a buffer; and (b) lyophilizing the mixture.

The lyophilized powder preferably is reconstituted by adding an aqueous solvent suitable for pharmaceutical administrations. Examples of suitable reconstitution solvents include, without limitation, water, saline, and phosphate buffered saline (PBS). Preferably, the lyophilized powder is reconstituted with normal (0.9%) saline. Upon reconstitution, an equilibrium is established between a boronate ester compound and compound (I). In some embodiments, equilibrium is reached quickly, e.g., within 10-15 minutes, after the addition of aqueous medium. The relative concentrations of a boronate ester compound and compound (I) present at equilibrium is dependent upon parameters such as, e.g., the pH of the solution, temperature, the nature of the boronic acid-complexing agent, and the ratio of boronic acid-complexing agent to a boronate ester compound present in the lyophilized powder.

The pharmaceutical compositions utilized in the present invention preferably are formulated for administration to a patient having, or at risk of developing or experiencing a recurrence of, systemic lupus erythematosus and/or lupus nephritis. Preferred pharmaceutical compositions utilized in the present invention are those formulated for oral, intravenous, or subcutaneous administration. Any of the above dosage forms containing a therapeutically effective amount of the compound of the present invention are well within the bounds of routine experimentation and within the scope of the present invention. In some embodiments, the pharmaceutical composition utilized in the present invention may further comprise the other therapeutic agent.

The amount of additional therapeutic agent present in a composition of this invention typically will be no more than the amount that would normally be administered in a composition comprising that therapeutic agent as the only active agent. Preferably, the amount of additional therapeutic agent will range from about 50% to about 100% of the amount normally present in a composition comprising that agent as the only therapeutically active agent.

In order that this invention be more fully understood, the following preparative and testing examples are set forth. These examples illustrate how to make or test specific compounds, and are not to be construed as limiting the scope of the invention in any way.

### EXAMPLES

### Example 1: Preparation of Compounds and Pharmaceutical Compositions

Compound (I) is prepared by methods disclosed in Olhava and Danca, U.S. Pat. No. 7,442,830, herein incorporated by reference in its entirety. The compound (Ia) is prepared by methods disclosed in Elliott et al., WO 2009/154737, herein incorporated by reference in its entirety. An oral capsule formulation of compound (Ia) is prepared by methods disclosed in Elliott et al., WO 2009/154737, herein incorporated by reference in its entirety. An IV formulation of compound (Ia) is prepared by methods disclosed in Elliott et al., WO 2009/154737, herein incorporated by reference in its entirety. A lyophilized formulation of compound (Ia) suitable for reconstitution into an IV formulation is prepared by methods disclosed in WO 2009/154737, herein incorporated by reference in its entirety. The compound (Ib) is prepared by methods disclosed in Elliott et al., WO 2009/154737, herein incorporated by reference in its entirety. An oral capsule formulation of compound (Ib) is prepared by methods disclosed in Elliott et al., WO 2009/154737, herein incorporated by reference in its entirety. An IV formulation of compound (Ib) is prepared by methods disclosed in Elliott et al., WO 2009/154737, herein incorporated by reference in its entirety. A lyophilized formulation of compound (Ib) suitable for reconstitution into an IV formulation is prepared by methods disclosed in WO 2009/154737, herein incorporated by reference in its entirety.

### Example 2

### Animal Experiment

F344 rats (male, 7 weeks old) were immunized with KLH plus adjuvant twice at day 0 and day 14. From 8 days after last immunization, rats were administered with vehicle or compound (I) (0.3 mpk, i.v. or 1.5 mpk, p.o.) twice weekly for 4 weeks, then the rats were sacrificed and serum, spleen and bone marrow were collected. All study protocols were approved by TAKEDA Animal Care and Use Committee.

### ELISA for Anti-KLH IgG

Serum was stored at −80° C. until use. Anti-KLH IgG titer in serum was measured by ELISA (KLH rat IgG ELISA kit, Shibayagi).

### ELISpot Assay

Splenocytes and bone marrow cells were collected from spleen and bone marrow. The cells were cultured for 2 hours on 96 well plate pre-coated with KLH (Sigma, H7017-20MG), then after washing with PBS, the plates were incubated with goat anti-rat IgG, HRP conjugate (Millipore, AP136P) for 1 hour. Anti-KLH antibody producing cells were visualized by adding TMB substrate and the number of anti-KLH antibody producing cells were counted by ELISpot reader system (AID).

### Statistic Analysis

Data represent mean values+s.e.m. (n=10). Student t test was used for statistical analysis (*p<0.05, **p<0.01 vs. control).

### Results

Four weeks treatment of KLH-immunized rats with Compound (I) (0.3 mpk, i.v. or 1.5 mpk, p.o.) significantly suppressed the anti-KLH IgG titer. Furthermore, Compound (I) suppressed the number of anti-KLH IgG-producing cells in spleen and bone marrow in ELISpot assay. Taken together, Compound (I) markedly suppressed the increase in anti-KLH IgG titer by depleting antibody-secreting cells in bone marrow and spleen in rat KLH-TDAR model.

| | | The number of | |
|---|---|---|---|
| | | anti-KLH IgG-secreting cells | |
| | Anti-KLH | bone marrow | Spleen |
| | IgG titer | (/3 × 10⁵ cells) | (/1 × 10⁶ cells) |
| Control | 770.6 ± 101.5 | 244.0 ± 42.2 | 114.8 ± 24.5 |
| Compound (I) | 509.5 ± 54.4* | 103.9 ± 16.9** | 37.0 ± 6.8** |
| 0.3 mpk, iv | | | |
| Compound (I) | 545.8 ± 67.0* | 156.4 ± 32.0 | 50.1 ± 12.2* |
| 1.5 mpk, po | | | |

Data are means and SE (n = 10)
*p < 0.05,
**p < 0.01 vs control (t-test)

While the foregoing invention has been described in some detail for purposes of clarity and understanding, these particular embodiments are to be considered as illustrative and not restrictive. It will be appreciated by one skilled in the art from a reading of this disclosure that various changes in form and detail can be made without departing from the true scope of the invention, which is to be defined by the appended claims rather than by the specific embodiments.

The patent and scientific literature referred to herein establishes knowledge that is available to those with skill in the art. Unless otherwise defined, all technical and scientific terms used herein have the same meaning as commonly understood by one of ordinary skill in the art to which this invention belongs. The issued patents, applications, and references that are cited herein are hereby incorporated by reference to the same extent as if each was specifically and individually indicated to be incorporated by reference. In the case of inconsistencies, the present disclosure, including definitions, will control.

This application is based on patent application No. 61/886,403 filed in USA, the contents of which are hereby incorporated by reference.

1. A method for the prophylaxis or treatment of systemic lupus erythematosus and/or lupus nephritis in a patient in need thereof, which comprises administering an effective amount of a compound which is [(1R)-1-({[(2,5-dichlorobenzoyl)amino]acetyl}amino)-3-methylbutyl]boronic acid or a citric acid ester thereof, or a pharmaceutically acceptable salt thereof to the patient.

2. The method of claim 1, wherein the compound is 2,2′-{2-[(1R)-1-({[(2,5-dichlorobenzoyl)amino]acetyl}amino)-3-methylbutyl]-5-oxo-1,3,2-dioxaborolane-4,4-diyl}diacetic acid, 4-(R,S)-(carboxymethyl)-2-((R)-1-(2-(2,5-dichlorobenzamido)acetamido)-3-methylbutyl)-6-oxo-1,3,2-dioxaborinane-4-carboxylic acid, or a mixture thereof.

3. The method of claim 1, wherein the compound is 2,2′-{2-[(1R)-1-({[(2,5-dichlorobenzoyl)amino]acetyl}amino)-3-methylbutyl]-5-oxo-1,3,2-dioxaborolane-4,4-diyl}diacetic acid.

4. The method of claim 1, wherein the compound is 4-(R,S)-(carboxymethyl)-2-((R)-1-(2-(2,5-dichlorobenzamido)acetamido)-3-methylbutyl)-6-oxo-1,3,2-dioxaborinane-4- carboxylic acid.

5. A compound which is [(1R)-1-({[(2,5-dichlorobenzoyl)amino]acetyl}amino)-3-methylbutyl]boronic acid or a citric acid ester thereof, or a pharmaceutically acceptable salt thereof, for use in the prophylaxis or treatment of systemic lupus erythematosus and/or lupus nephritis.

6. Use of a compound which is [(1R)-1-({[(2,5-dichlorobenzoyl)amino]acetyl}amino)-3-methylbutyl]boronic acid or a citric acid ester thereof, or a pharmaceutically acceptable salt thereof, for preparation of a medicament for the prophylaxis or treatment of systemic lupus erythematosus and/or lupus nephritis.

7. A medicament for the prophylaxis or treatment of systemic lupus erythematosus and/or lupus nephritis, wherein the medicament comprises a compound which is [(1R)-1-({[(2,5-dichlorobenzoyl)amino]acetyl}amino)-3-methylbutyl]boronic acid or a citric acid ester thereof, or a pharmaceutically acceptable salt thereof.
